(19) United States
(12) Patent Application Publication
(10) Pub. No.: US 2025/0339002 A1
(43) Pub. Date: Nov. 6, 2025

(54) Finger-Wipe Apparatus

(71) Applicants: Michael W. Hall, Oklahoma City, OK (US); Tanner Heatley, Oklahoma City, OK (US); Jeremy Ussery, Renton, WA (US)

(72) Inventors: Michael W. Hall, Oklahoma City, OK (US); Tanner Heatley, Oklahoma City, OK (US); Jeremy Ussery, Renton, WA (US)

(21) Appl. No.: 18/654,161

(22) Filed: May 3, 2024

(51) Int. Cl. A47K 7/03; A61L 2/00

(52) U.S. Cl. CPC A47K 7/03; A61L 2/0088; A61L 2202/15; A61L 2202/17

(57) ABSTRACT

An apparatus for providing a means of wiping human fingers or a portion of a hand comprises a top component, a body component, and a base component interconnected for functionality. The top component is configured to allow wiping of the fingers or hand; the outer surface of the top component includes a fabric, with which the fingers or hand are in contact when in use. The body component includes a spray bottle containing a hand sanitizing or other type of antimicrobial solution used to assist in the skin cleaning process, such that in operation the solution sprays into and through the medium of the top component and to the skin. The body component may also include an outer fabric sleeve for drying of the skin when needed. The base component provides stability for the overall apparatus in addition to providing a means of catching falling particles resulting from usage.

### FIELD OF THE INVENTION

The present invention relates to the field of personal skin hygiene, particularly finger or hand cleansing. Specifically, aspects of the invention comprise a part for allowing the fingers or hand to be wiped, a part for dispensing of a hand sanitizing fluid or other antimicrobial solution toward the skin, and a part for holding the apparatus upright and simultaneously collecting food crumbs and other particles as they fall.

### BACKGROUND OF THE INVENTION

A person at a desk workstation or using gaming controllers or other electronic devices may occasionally snack on food during breaks. This results in food crumbs and grease from food, as well as various germs, culminating on the skin of the fingers and other parts of the hand. A quick and effective way to wipe the skin clean of these unwanted particles and germs may be desired, before continuing to contact the pertinent electronic device such as a keyboard, mouse, or controller. The invented apparatus provides the means for a user to quickly and conveniently clean crumbs, grease, and the like from fingers or other parts of the hand, which prolongs the usable life of electronic devices to be handled subsequently.

Traditionally, when one has been snacking at a workstation or during a break from playing electronic games, the finger cleaning options before resuming the work or play have been to (A) use paper towels, (B) move to another room to wash the hands, (C) wipe the hands on clothes, or (D) do nothing by leaving the food crumbs on the skin while handling the electronic devices such as a keyboard, mouse, or controller. Usage of paper towels (A) creates waste ultimately to be handled at a landfill. Moving to another room or to a sink (B), takes more time than may be desired by the user. Wiping food crumbs and grease onto one's clothes (C), can result in one's clothing appearance being less professional, and can stain or otherwise damage the clothes in the long term. Doing nothing (D), can result in the electronic devices becoming impaired over time, shortening their usable life.

In situations that aren't associated with handling electronic devices, such as a cashier handling cash, the user may also wish to quickly wipe the fingers clean from time to time throughout the shift or day.

A need exists to utilize the effectiveness of cleaning fingers using an appropriate type of “rinseless” hand sanitizing fluid or other antimicrobial solution combined with a fabric material designed to rub the skin free of unwanted particles and germs. When further considering the time savings and convenience associated with not having to leave a workstation or gaming console to wipe fingers clean, the present invention offers a resolution to the user. Usage of the invention can also decrease paper towel waste, spare one's clothes from a dirty appearance, and preserve electronic devices.

### SUMMARY OF THE INVENTION

The present invention relates to an apparatus comprising an assembly of components that can be used to clean the fingers. Accordingly, the object of this invention is to provide a convenient means for a person to wipe their fingers clean of crumbs or other unwanted particles. The apparatus comprises a top component, a body component, and a base component interconnected for functionality.

The top component is configured to allow wiping of the fingers or hand; the outer surface of the top component includes a fabric, on which surface the fingers or hand are rubbed to remove crumbs or other unwanted particles. The interior portion of this top component may consist of a porous, yet firm foam material, that allows flow of the liquid when sprayed upward to pass through to the exterior surface and through the outer fabric to the skin, while simultaneously providing the rigidity necessary to transfer force in order to depress the dispenser below. The interior foam subcomponent is manufactured with a hollowed core at its bottom end, configured to allow attachment to the top pump dispenser portion of the body component below. The exterior cleaning fabric may be made to be removable, to allow it to be occasionally laundered.

The body component comprises a spray bottle containing a hand sanitizing or other type of antimicrobial solution used to assist in the skin cleaning process, such that in operation the solution sprays into and through the medium of the top component. The spray bottle comprises a reservoir subcomponent containing the desired cleaning solution, and a pump dispenser subcomponent at the top end of the bottle. The pump dispenser is configured to spray the solution upward through several small holes in the top part that is depressed downward to pump the liquid upward. Additionally, the top piece of the pump dispenser, typically pressed downward to activate upward movement of the fluid, is configured to a shape that allows transfer of the downward depression force from the medium of the top component when the user presses downward; this shape may be that of a flat disc of a diameter sufficient to function properly with the medium material of the top component. The body component may also include an outer fabric sleeve, the fabric material selected being suitable for drying of the skin when desired by the user.

The base component comprises a dish-shaped base to which the bottom end of the bottle of the body component attaches. The base component provides stability for the overall apparatus in addition to providing a means of catching any falling crumbs or particles resulting from the wiping action of the user.

### BRIEF DESCRIPTION OF THE DRAWINGS

FIG. 1 is a perspective view of the first embodiment of the present invention, showing the exterior subcomponents. The user wipes the hand clean on the top component and may press downward on it to dispense antimicrobial solution toward the skin for further cleaning.

FIG. 2 is an exploded side view showing the subcomponents of the first embodiment, and the conceptual assembly composition of the subcomponents.

FIG. 3 shows perspective views of additional embodiments, illustrating that the top component may be manufactured in various shapes.

FIG. 4 shows side views of additional embodiments of the present invention, illustrating that the bottle and its sleeve may be manufactured in various shapes and styles to appeal to more users or for ergonomic reasons.

FIG. 5 depicts a portion of the body component, particularly the figure is an exterior top view and a side cross-sectional view through the centerline of the holes of the head subcomponent of the spray dispenser pump, showing one embodiment of the hole pattern in the top portion of the head surface to direct and discharge the liquid generally upward when the head is depressed downward.

FIG. 6 shows a perspective view of another potential embodiment, further illustrating that the components can vary in shape and configuration to suit users' preferences.

### DETAILED DESCRIPTION OF THE INVENTION

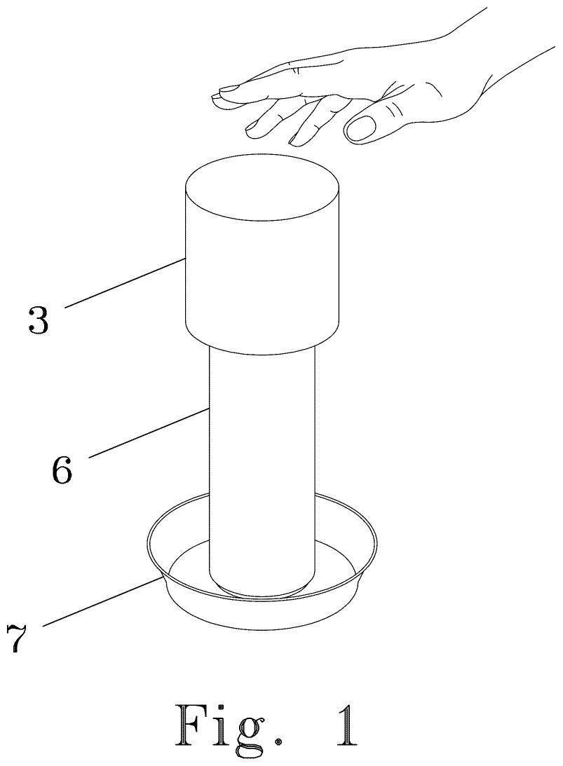

The invention disclosed herein relates to an apparatus intended to provide a convenient way for the user to wipe the fingers clean of food crumbs or other undesirable particles. The apparatus design is envisioned to be placed on desktops or tabletops, or other locations convenient for use. FIG. 1 depicts one embodiment manufactured in accordance with the present invention, comprising a top component for wiping, a body component including a reservoir or bottle containing sanitizing or antimicrobial liquid together with a compatible spray pump dispenser, and a base component to stabilize the assembly and catch wiped particles. Generally, these components of the apparatus can have various shapes, dimensions, and styles. The exterior subcomponents of these components are labeled in the figure.

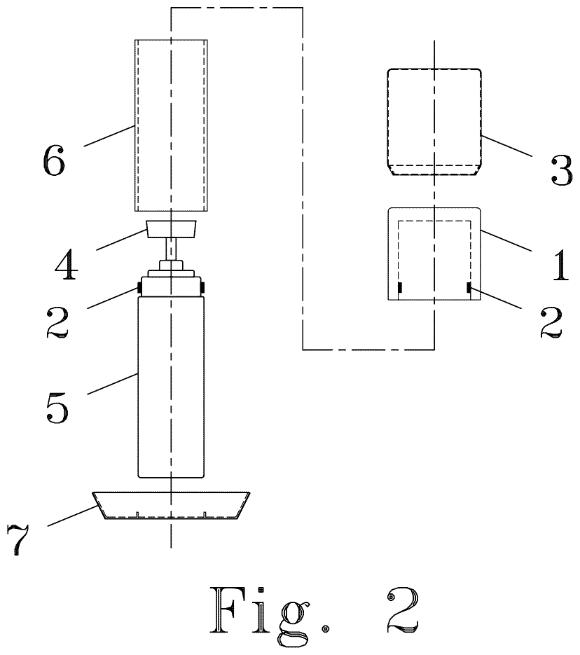

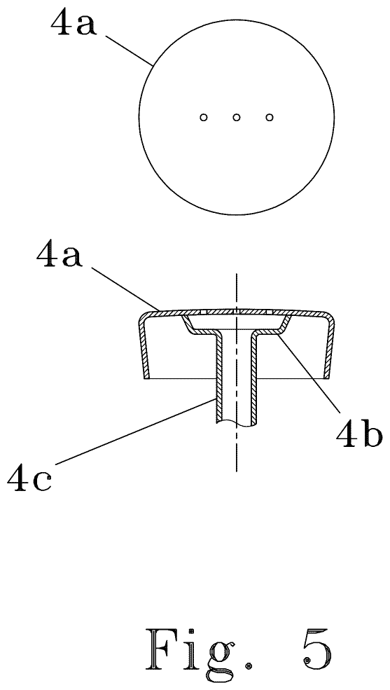

In the embodiment depicted in FIG. 2, main subcomponents are shown disassembled for clarity. The exterior fabric cover 3 of the top component is sized and shaped to fit snugly on and around the interior foam subcomponent 1 and is made to be easily removable for washing. The fabric of 3 may consist of any preferred textile such as microfiber, terrycloth, or a polyester and spandex blend, generally to be selected for desired moisture wicking properties, ability to abrade food crumbs and other particles from the skin, and softness to the touch. The bottom end of the cover is secured to the interior foam subcomponent, such as with an elastic band or tie string, in a manner that allows easy removal and reinstallation. Subcomponent 1 is a permeable foam material such as a reticulated polyurethane foam, manufactured to the desired size and shape for the preferred embodiment. The lower interior portion of 1 is hollow to conform to the top dimensions of the spray dispenser pump 4; the upper portion above the top surface of 4 remains to provide a soft feel to the top of the top component. The functional requirements of 1 are that it be a material sufficiently porous to allow the discharged liquid to permeate through the medium rapidly, and be sufficiently firm to transfer the downward force exerted by the user when wishing to depress the spray dispenser pump head 4 to dispense the antimicrobial liquid. Attachment of the lower portion of 1 to the upper portion of reservoir 5 is accomplished with Velcro 2 or similar means, that secures the top component to the body component yet permits occasional disassembly. Reservoir 5 is sized and dimensioned to contain multiple doses of the sanitizing or antimicrobial liquid. In a preferred embodiment, reservoir 5 may contain from about 300 mL to 500 ml of solution, and is configured similar to a typical bottle of shampoo, body wash, or other liquid commonly found in stores, with spray pump dispenser 4 integral to the bottle and attached thereto with a screw-on lid. This embodiment further illustrates the head subcomponent of dispenser 4 has a relatively flat and wide top surface in order to allow the user's hand to depress it through the top portion of 1 without puncturing the foam material. FIG. 5 shows one embodiment of the head portion 4a of dispenser 4, illustrating that holes for discharging the sanitizing or antimicrobial solution are in the top surface, such that when the dispenser is depressed, the liquid sprays upward into and through the media of 1 and 3 to reach the skin for cleaning purposes. The spray dispenser pump 4 functions the same as a typical dispenser commonly found on a bottle of shampoo, body wash, or other liquid, with the liquid being pressurized to move upward through an axial passage tube 4c and into lateral passage tube 4b and then discharged through the exterior holes. Although this embodiment depicts three discharge holes located in the top surface of head 4a, other hole quantities, arrangements, and configurations are possible. FIG. 2 portrays an embodiment in which an exterior sleeve 6 covers the outside surface of reservoir 5, and the top portion of spray pump dispenser 4 is concealed inside 1. Sleeve 6 is configured to fit snugly around reservoir 5, and may be made of neoprene foam or a flexible material functionally suitable for drying the skin as well as for gripping the apparatus for carrying purposes. The base component 7 may have a dish shape and has a flat bottom surface, sufficiently wide to provide stability for the apparatus as well as to catch falling particles rubbed from the skin. The base component 7 may be made of a rigid plastic material such as polypropylene or similar material, such that it would provide structure to the apparatus. Soft pads may be added to the bottom surface of 7 if the material used is of hardness greater than typical wood furniture, to prevent scratching of the furniture. The bottom end of reservoir 5 is attached to the center of 7 either in a mechanical manner or by fusing them together during manufacture; they need not be made to separate.

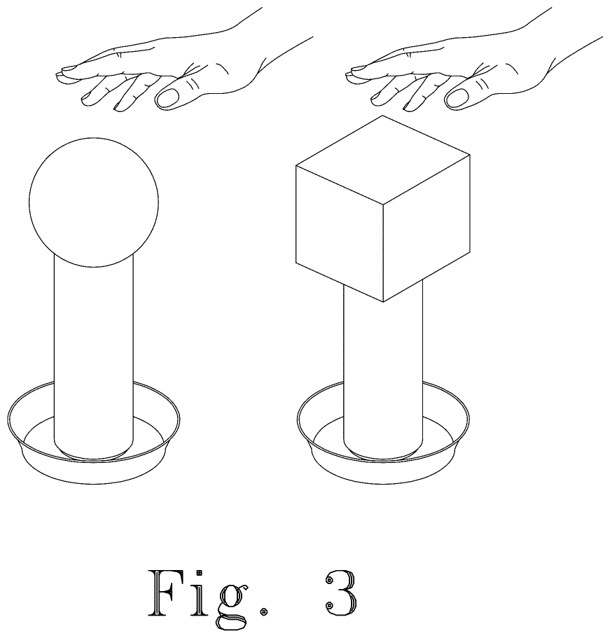

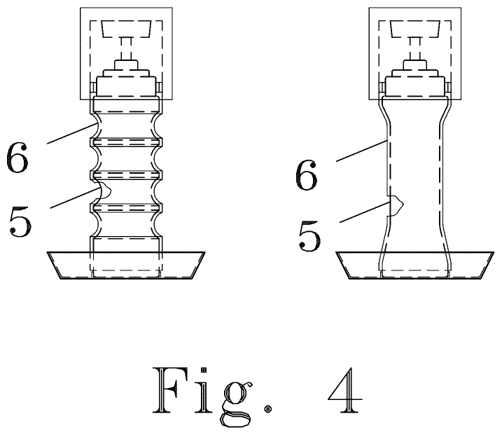

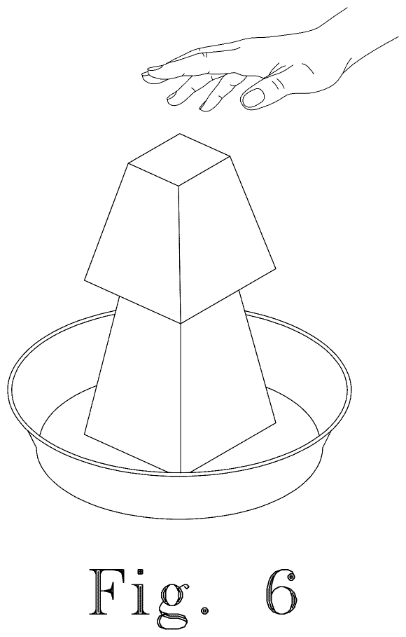

It is recognized that the apparatus may be made in various shapes, sizes, and styles to appeal to various prospective users. FIG. 3 and FIG. 4 both offer possible alternative shapes for the top component and body component embodiments, respectively. FIG. 6 further depicts possible alternative shapes for the components. While this invention is satisfied by embodiments depicted herein, the present disclosure is to be considered exemplary of the principles of the invention and is not intended to limit the invention to only the embodiments illustrated and described. Numerous variations may be made without departure from the spirit of the invention.

What is claimed is:

1. An apparatus for cleaning skin of the fingers or part of the hand after snacking or handling of unclean objects, said apparatus comprising:
a. a top unit, the exterior of which comprises a cloth, microfiber or other fabric material suitable for contact with human skin for wiping or cleaning purposes, and the interior of which comprises a porous and yielding material such as a suitable open-cell foam;
b. a body unit, connecting to both the top unit the base unit, comprising an integrated hand sanitizing spray mist dispenser;
c. a base unit designed to allow the apparatus to sit near a workstation, on a coffee table, or other surface deemed convenient to the user.

2. The apparatus of claim 1 wherein the exterior fabric of the top unit is a removable cloth, microfiber, or other exterior material for easy cleaning and replacement.

3. The apparatus of claim 1 wherein the top unit is designed to be aesthetically pleasing and customizable with different designs, styles, patterns, or shapes, to appeal to individual tastes of the user.

4. The apparatus of claim 1 wherein a user-activated mechanism such as an actuator or button is located in the top unit and integrated with the hand sanitizing spray dispenser of the body unit and may be depressed to dispense the hand sanitizing spray mist onto the cloth, microfiber, or other exterior fabric from within the top unit for purposes of sanitizing the skin of the user.

5. The apparatus of claim 1 wherein the hand sanitizing spray liquid is stored in a refillable container of the body unit, allowing for easy refilling by the user.

6. The apparatus of claim 1, designed to save time for the user and provide a convenient way to clean skin of the fingers or part of the hand without the need to leave the workstation, coffee table area, or other conceived usage locations.

7. The apparatus of claim 1, designed to prevent the spread of germs and bacteria by providing a quick and convenient way for the user to clean their fingers or hands after snacking or handling unclean objects.

8. The apparatus of claim 1, designed to prolong the life of keyboards, controllers, or other electronic devices by providing a way to remove food crumbs or other foreign particles from the skin that could otherwise damage the electronic components by contact.

9. The apparatus of claim 1, designed to be an ornamental item for the workplace or home, enhancing the aesthetics of the surrounding work or home environment or to the tastes of the individual user.

10. The apparatus of claim 1, designed to reduce excess refuse such as paper towels by providing an alternative way to clean the hands with a washable fabric.
